(19) United States
(12) Patent Application Publication
(10) Pub. No.: US 2023/0408502 A1
(43) Pub. Date: Dec. 21, 2023

(54) MAGNETIC PARTICLE AND PARTICLE FOR IMMUNOLOGICAL TEST

(71) Applicants: CANON KABUSHIKI KAISHA, Tokyo (JP); CANON MEDICAL SYSTEMS CORPORATION, Tochigi (JP)

(72) Inventors: DAISUKE SASAGURI, Kanagawa (JP); SATOSHI YAMABI, Kanagawa (JP); NORISHIGE KAKEGAWA, Tokyo (JP); SHIGEMOTO ABE, Kanagawa (JP); MINAKO NAKASU, Tokyo (JP)

(21) Appl. No.: 18/329,748

(22) Filed: Jun. 6, 2023

(30) Foreign Application Priority Data
Jun. 17, 2022 (JP) 2022-098318
May 26, 2023 (JP) 2023-086755

(51) Int. Cl. G01N 33/543; B82Y 5/00

(52) U.S. Cl. CPC G01N 33/5434; B82Y 5/00

(57) ABSTRACT

Provided is a magnetic particle having high magnetic field responsiveness in detection of a substance to be measured, such as an antigen or an antibody, from a specimen. The magnetic particle includes a magnetic core particle and a polymer layer arranged on a surface of the magnetic core particle. The magnetic core particle contains an aggregation of a plurality of magnetic nanoparticles. The polymer layer contains a polymer having at least one kind of functional group selected from the group consisting of: a carboxyl group; an amino group; a thiol group; an epoxy group; a maleimide group; and a succinimidyl group.

### BACKGROUND OF THE INVENTION

### Field of the Invention

The present invention relates to a magnetic particle and a particle for an immunological test.

### Description of the Related Art

In recent years, a magnetic particle has been used for a wide variety of applications. For example, as a bioapplication, there is given a usage example in which an antigen, an antibody, or the like in a specimen substance is captured or separated. In addition, the magnetic particle is used for a specimen test to be used in diagnosis. Specifically, there is given a method of detecting an antigen (antibody) from a specimen through use of a magnetic particle having an antibody (antigen), which specifically binds to the antigen (antibody), bound thereto and a sensor having an antibody (antigen), which specifically binds to the antigen (antibody), immobilized thereon. The magnetic particle is used as a test reagent for determining positivity or negativity through identification of the magnetic particle having such antibody (antigen) bound thereto based on light, a color, or the like. Such magnetic particle is generally called a magnetic particle for bioseparation.

Such magnetic particle has been improved so as to exhibit high magnetic field responsiveness in order to manipulate the particle with an external magnetic field. For example, there have been performed an increase in amount of a magnetic material incorporated into a magnetic material particle, and inclusion of a magnetic material showing high magnetization.

As such magnetic particle for bioseparation, for example, there is a proposal of a magnetic particle obtained by incorporating magnetic nanoparticles into a polymer matrix (see Japanese Patent Application Laid-Open No. 2006-292721).

In addition, as the magnetic particle for bioseparation, there is also a proposal of a structure in which magnetic nanoparticles are incorporated into a silica matrix (see Japanese Patent Application Laid-Open No. 2013-19889).

Further, there is also a proposal of a particle structure in which a polymer layer is formed as a surface layer of a single magnetic particle (see Japanese Patent Application Laid-Open No. 2012-177691).

The magnetic particle for bioseparation is captured by applying an external magnetic field under a state in which the particle is dispersed in a solution. In order to cause the particle to move quickly, it is required that the magnetic material be incorporated into each individual particle at a high density. The inventors investigated a capture speed of a particle equivalent to the structure described in Japanese Patent Application Laid-Open No. 2006-292721 and Japanese Patent Application Laid-Open No. 2013-19889, in which the magnetic nanoparticles were incorporated into the polymer matrix or the silica matrix, by applying a magnetic field to the particle. The capture speed was low, and sufficient performance was not obtained. In addition, when a similar operation was performed on the particle of the structure described in Japanese Patent Application Laid-Open No. 2012-177691, although the capture speed was increased, once captured, the particle was not able to be easily redispersed owing to residual magnetization of the particle even after the applied magnetic field was stopped, and hence subsequent treatment was difficult. The above-mentioned investigations were performed using a test apparatus including an optical waveguide-type system.

Accordingly, an object of the present invention is to provide a magnetic particle having a high magnetic material content and being free of residual magnetization, the magnetic particle enabling quick collection of a substance of interest from a specimen solution through application of a magnetic field, and being capable of being quickly redispersed after the application of the magnetic field is stopped.

### SUMMARY OF THE INVENTION

The present invention relates to a magnetic particle including: a magnetic core particle; and a polymer layer arranged on a surface of the magnetic core particle, wherein the magnetic core particle contains an aggregation of a plurality of magnetic nanoparticles, and wherein the polymer layer contains a polymer having at least one kind of functional group selected from the group consisting of: a carboxyl group; an amino group; a thiol group; an epoxy group; a maleimide group; and a succinimidyl group.

The present invention also relates to a particle for an immunological test including: the magnetic particle; and a ligand, wherein the ligand and the functional group have a chemical bond therebetween.

Further features of the present invention will become apparent from the following description of exemplary embodiments with reference to the attached drawings.

### BRIEF DESCRIPTION OF THE DRAWINGS

FIG. 1 is a schematic view for illustrating a configuration of a magnetic particle according to an embodiment of the present invention.

FIG. 2 is an SEM observation image of magnetic core particles according to the embodiment of the present invention.

### DESCRIPTION OF THE EMBODIMENTS

An embodiment of the present invention is described in detail below. Various physical property values are values at 25° C. unless otherwise stated. The number average particle diameter of a magnetic particle refers to a number average particle diameter of the magnetic particle also including a polymer layer arranged on the surface of the magnetic particle. Now, as a substance to be measured to be detected from a specimen, an antigen is described as an example.

A magnetic particle according to one embodiment of the present invention is a magnetic particle including: a magnetic core particle; and a polymer layer arranged on a surface of the magnetic core particle, wherein the magnetic core particle contains an aggregation of a plurality of magnetic nanoparticles, and wherein the polymer layer contains a polymer having at least one kind of functional group selected from the group consisting of: a carboxyl group; an amino group; a thiol group; an epoxy group; a maleimide group; and a succinimidyl group.

In the collection of a substance of interest (e.g., an antigen) from a specimen solution, a magnetic particle having an antibody, which specifically binds to the antigen, bound thereto is used. Specifically, the magnetic particle having the antibody, which specifically binds to the antigen, bound thereto is placed in a container having placed therein the specimen solution containing the antigen to allow the magnetic particle to capture the antigen in the specimen solution. The substance of interest can be separated from the specimen solution by collecting the magnetic particle that has captured the antigen with a magnet from the outside of the container and removing a supernatant solution.

Hitherto, the magnetic particle to be used for the capture of the substance of interest has not had a sufficient content of a magnetic material component incorporated into the magnetic particle, and hence a magnetic force acting on the magnetic particle has been small. Accordingly, the collection of the magnetic particle with a neodymium magnet or the like took time, and the operation of separating the substance of interest took time.

In view of the foregoing, in the present invention, it has been conceived that an increase in content of the magnetic material in the magnetic particle is important for enabling the magnetic particle to quickly move with a magnetic field. In the present invention, as an approach to improving the content of the magnetic material, it is required that the magnetic core particle contain an aggregation of magnetic nanoparticles.

It is preferred that the magnetic nanoparticles according to the present invention be superparamagnetic, and have a number average particle diameter of 20 nm or less. In addition, in particular, a magnetic particle having a small particle diameter is preferably used because the specific surface area of the particle can be increased to increase the probability of capturing an antigen, and hence both of the ability to capture the substance of interest and the collection speed of the magnetic particle can be improved as compared to the related-art magnetic particle structure.

In addition, in the present invention, the magnetic particle is a magnetic particle including: a magnetic core particle; and a polymer layer arranged on a surface of the magnetic core particle. Further, it is required that the polymer layer contain a polymer having at least one kind of functional group selected from the group consisting of: a carboxyl group; an amino group; a thiol group; an epoxy group; a maleimide group; and a succinimidyl group. By having any such functional group, the magnetic particle can be bonded to an antibody, and hence can effectively capture an antigen.

(Outline of Configuration of Magnetic Particle according to This Embodiment)

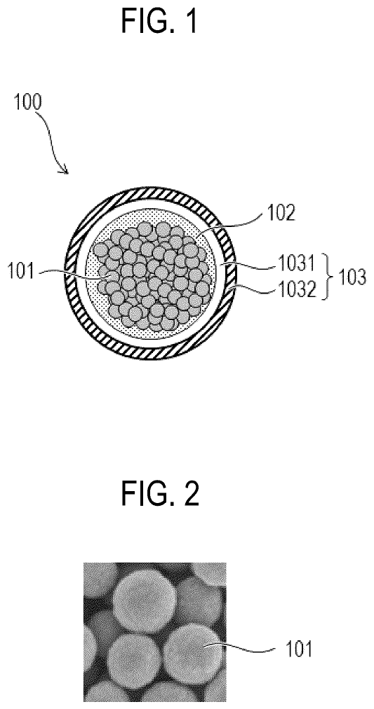

As illustrated in FIG. 1, a magnetic particle 100 according to this embodiment includes a magnetic core particle 101 formed of an aggregation of magnetic nanoparticles, and an inorganic coating layer 102 arranged on the surface of the magnetic core particle 101, and includes a polymer layer 103 formed on the inorganic coating layer 102. The polymer layer 103 is sometimes formed of two kinds of materials, and in that case, includes a polymer layer 1031 (first polymer layer) and a polymer layer 1032 (second polymer layer) in order of closeness to the magnetic core particle 101. The polymer layer 1032 contains a functional group (not shown) to which a ligand can be bonded. The magnetic core particle 101 of the magnetic particle 100 has a structure in which a plurality of magnetic nanoparticles are associated substantially alone. With this structure, the proportion of a magnetic component can be increased as compared to the structure in which magnetic nanoparticles are dispersed in a medium using a nonmagnetic material, such as a polymer or ceramics, as a matrix component as in Japanese Patent Application Laid-Open No. 2006-292721, Japanese Patent Application Laid-Open No. 2013-19889, and Japanese Patent Application Laid-Open No. 2012-177691. As a result, the magnetic particle of the present invention can be enhanced in magnetic field responsiveness as compared to the related-art structure.

<Magnetic Particle>

The following case is preferred: the magnetic particle of the present invention has a structure including: a magnetic core particle containing an aggregation of magnetic nanoparticles; an inorganic layer of a nonmagnetic material formed as a surface layer on the magnetic core particle; and a polymer layer formed via the inorganic layer. In the present invention, the external appearance of the magnetic core particle may be observed with a scanning electron microscope (SEM). In addition, the thickness of a nonmagnetic layer, i.e., the inorganic layer or the polymer layer may be observed with a transmission electron microscope (TEM). In the case of dry particles, the number average particle diameter thereof may be determined from the average value of the long diameters of 100 particles through SEM observation.

The number average particle diameter of the magnetic particle of the present invention is not particularly limited, but in consideration of, for example, responsiveness to an applied magnetic field, is preferably 0.1 μm or more and 2.0 μm or less. A case in which the number average particle diameter is less than 0.1 μm is not preferred because the work of collecting the magnetic particle with a magnet or the like takes too much time.

<Magnetic Core Particle>

The magnetic particle of the present invention has a feature in that the magnetic core particle contains an aggregation of magnetic nanoparticles each having magnetism. The magnetic material refers to a material that is magnetized when a magnetic field is applied thereto. The magnetic nanoparticles each preferably contain at least one kind selected from the group consisting of: a metal; and a metal oxide. Examples of the metal include iron, manganese, nickel, cobalt, and chromium. Examples of the metal oxide include magnetic iron oxides, such as magnetite (Fe₃O₄), maghemite (γ-iron(III) oxide) (γ-Fe₂O₃), and ferrite. Of those, from the viewpoints of, for example, large magnetization and stability in a solution, a case in which the magnetic nanoparticles are each formed of at least any one of magnetite (Fe₃O₄) or γ-iron(III) oxide (γ-Fe₂O₃) is preferred. In addition, magnetite fine particles having a number average particle diameter of 20 nm or less are preferred because the particles have large saturation magnetization and are a superparamagnetic material, thereby having small residual magnetization.

Herein, the “magnetization” refers to a phenomenon in which the magnetic material is polarized to have a magnetic moment when an external magnetic field is applied to the magnetic material, and the “saturation magnetization” refers to a value at which the magnetization that is increased with the intensity of the magnetic field is saturated. The saturation magnetization may be adjusted based on, for example, the particle diameter of the magnetic nanoparticles. In addition, the “residual magnetization” refers to the magnetization that remains in the magnetic material when the magnetic field is rendered zero after the external magnetic field is applied to the magnetic material. The residual magnetization of the magnetic particle of the present invention is more preferably zero.

(Method of Synthesizing Magnetic Core Particle)

A method of forming the magnetic core particle that is formed of iron oxide, in particular, an aggregation of magnetite nanoparticles is described. A raw material for the magnetite nanoparticles may be selected from, for example, iron chlorides (FeCl₂ and FeCl₃), iron nitrate (Fe(NO₃)₂), iron sulfate (FeSO₄), and hydrates thereof (FeCl₂·4H₂O, FeCl₃·6H₂O, Fe(NO₃)₂·6H₂O, and FeSO₄·7H₂O), and is particularly preferably selected from iron chlorides (FeCl₂ and FeCl₃) and hydrates thereof (FeCl₂·4H₂O and FeCl₃·6H₂O), but a plurality of iron compounds may be used as a mixture.

As an example, a production method using iron(III) chloride hydrate (FeCl₃.6H₂O) is described. Iron(III) chloride hydrate is dissolved in ethylene glycol, which is a high-boiling-point solvent, by being thoroughly stirred. To the solution, sodium acetate, polyethylene glycol (PEG), or the like is added as an additive to prepare a raw material solution. The raw material solution is placed in a container made of glass, and is sealed, together with the container made of glass, in a pressure-resistant container including an internal cylinder made of Teflon (trademark).

After that, the pressure-resistant container is heated in a thermostatic chamber set to a temperature of from 170° C. to 190° C., which is lower than the boiling point of the solvent. As a result, an aggregation of magnetite nanoparticles is formed in the solvent. The solvent is removed, and the residue is thoroughly washed with an alcohol and water, and then dried at 60° C. As a result, a magnetite aggregation is obtained. The mode of heating is not limited to the thermostatic chamber, and various methods such as an oil bath may be adopted.

The obtained magnetite aggregation may be recognized by observation with an SEM to have a structure in which nanoparticles are associated. In addition, it may be recognized by X-ray diffractometry (XRD) that the crystal structure of the aggregation is magnetite.

In consideration of, for example, responsiveness to an applied magnetic field, the number average particle diameter of the magnetite aggregation is preferably 0.05 μm or more and 1.95 μm or less. The number average particle diameter of the magnetic core particle may be measured by the same method as that for the magnetic particle described above.

The thus produced magnetic core particle can have a higher content of the magnetic nanoparticles than the related-art magnetic particle having the magnetic nanoparticles dispersed in a polymer or silica, and hence is improved in responsiveness to an applied magnetic field.

<Surface Layer Formation>

The magnetic particle of the present invention includes the polymer layer arranged on the surface of the magnetic core particle. Herein, the expression “includes the polymer layer arranged on the surface of the magnetic core particle” means a case in which the polymer layer is directly arranged on the surface of the magnetic core particle, or a configuration in which the polymer layer is arranged on the magnetic core particle via a layer such as a silica layer to be described later. The magnetic particle of the present invention preferably has a configuration of having a surface layer formed of a polymer.

(Formation of Inorganic Coating Layer)

The magnetic core particle that is the aggregation of the magnetic nanoparticles is an aggregate of nanoparticles, and hence has fine irregularities on the surface thereof. In order to alleviate the irregularities, an inorganic coating layer is preferably arranged. A material therefor is not limited, but silica is preferred as a material capable of stably forming a layer. A silica layer may be formed on the surface of the magnetic core particle by, for example, dispersing the magnetic core particle in a mixed solvent of water and an alcohol such as ethanol, and adding tetraethoxysilane (TEOS) and ammonia water serving as a catalyst.

The thickness of the silica layer is not particularly limited, but in consideration of, for example, responsiveness to an applied magnetic field, is preferably 5 nm or more and 200 nm or less, more preferably 10 nm or more and 100 nm or less.

(Formation of Polymer Layer)

In the following description, the term “(meth)acrylate” means “acrylate or methacrylate.”

The surface layer of the polymer has a functional group to which an antibody can be bonded. The functional group to which an antibody can be bonded is preferably at least one kind selected from the group consisting of: a carboxyl group; an amino group; a thiol group; an epoxy group; a maleimide group; and a succinimidyl group.

In the formation of the polymer layer, it is preferred to use: a monomer having a carboxyl group, such as (meth)acrylic acid; a monomer having an amino group, such as (meth)acrylamide; a monomer having an epoxy group, such as glycidyl (meth)acrylate; and a monomer having a succinimidyl group, such as N-succinimidyl acrylate.

In addition to the above-mentioned monomers, (meth)acrylates each having a hydrophilic group, such as glycerol (meth)acrylate, 2-hydroxyethyl (meth)acrylate, methoxyethyl (meth)acrylate, and polyethylene glycol mono(meth)acrylate; styrenes, such as styrene, p-chlorostyrene, and α-methylstyrene; and the like may also be used. In order to suppress non-specific adsorption to the magnetic particle, it is preferred to use (meth)acrylates each having a hydrophilic group.

The polymer layer may be crosslinked as required. The crosslinking of the polymer layer is effective when the magnetic particle is used in a specimen in which nonspecific adsorption may occur. As the crosslinking agent, for example, a hydrophilic crosslinking agent and a hydrophobic crosslinking agent that are generally used may be selected. When a hydrophobic crosslinking agent is adopted, examples thereof include divinylbenzene, ethylene glycol diacrylate, ethylene glycol dimethacrylate, trimethylolpropane triacrylate, trimethylolpropane trimethacrylate, pentaerythritol triacrylate, pentaerythritol trimethacrylate, dipentaerythritol hexaacrylate, and dipentaerythritol hexamethacrylate. In addition, when a hydrophilic crosslinking agent is adopted, examples thereof include polyethylene glycol diacrylate, polyethylene glycol dimethacrylate, and a poly(meth)acrylic ester of polyvinyl alcohol.

When treatment with a silane coupling agent is added as surface preparation in the formation of such polymer layer, the polymer layer can be homogeneously formed. The kind of the silane coupling agent is not particularly limited, but 3-methacryloxypropyltrimethoxysilane (LS-3380, manufactured by Shin-Etsu Chemical Co., Ltd.) or the like is generally used.

The polymer layer is preferably formed of two kinds of layers. The polymer of a first layer may be formed in advance as an underlayer of the above-mentioned polymer layer. In this case, when the polymer layer that is the first layer is referred to as “first polymer layer”, and the polymer layer that is the second layer is referred to as “second polymer layer”, a preferred configuration is as follows: the second polymer layer serves as the outermost surface layer of the magnetic particle. The first polymer layer may be arranged to protect the core particle of the magnetic nanoparticles. A hydrophobic polymer is preferred as the first polymer layer, and it is preferred to use, for example, a styrene, such as styrene, p-chlorostyrene, or α-methylstyrene. In addition, the second polymer layer formed on the first polymer layer and serving as the outermost surface layer of the magnetic particle preferably contains a polymer having a unit derived from glycidyl methacrylate. The first polymer layer may also be crosslinked as with the outermost surface polymer layer that is the second polymer layer. The kind of the crosslinking agent is not particularly limited, and a hydrophobic crosslinking agent is used for the purpose of protection. Examples thereof include divinylbenzene, ethylene glycol diacrylate, ethylene glycol dimethacrylate, trimethylolpropane triacrylate, trimethylolpropane trimethacrylate, pentaerythritol triacrylate, pentaerythritol trimethacrylate, dipentaerythritol hexaacrylate, and dipentaerythritol hexamethacrylate.

When the magnetic particle is used in an application where a biologically derived substance of interest (specimen) is detected, aggregation due to a component derived from the specimen other than the substance of interest (nonspecific aggregation) sometimes occurs, but the crosslinking of the polymer layer that is the outermost surface layer of the particle can suppress the nonspecific aggregation, and hence is preferred.

The thickness of the polymer layer is not particularly limited, but in consideration of, for example, responsiveness to an applied magnetic field, the thickness of the first polymer layer is preferably 5 nm or more and 100 nm or less, and the thickness of the second polymer layer is preferably 5 nm or more and 200 nm or less.

In addition, the functional group to which an antibody can be bonded may be added after the polymerization of the polymer. For example, when a reagent having a carboxyl group, such as mercaptopropionic acid or mercaptosuccinic acid, is allowed to act on a polymer layer having a glycidyl group, the carboxyl group can be introduced into the polymer layer. In addition, a maleimide group may be introduced by adding N-(2-hydroxyethyl)maleimide to the polymer obtained by polymerization.

<Ligand>

The “ligand” refers to a compound that specifically binds to a receptor of a particular target substance. The ligand binds to the target substance at a predetermined site and has selectively or specifically high affinity. Examples thereof include: an antigen and an antibody; an enzyme protein and a substrate thereof; a signal substance typified by a hormone or a neurotransmitter and a receptor thereof; nucleic acids; and avidin and biotin, but are not limited thereto to the extent that the purpose of the above-mentioned embodiment can be achieved. Specific examples of the ligand include an antigen, an antibody, an antigen-binding fragment (e.g., Fab, F(ab′)₂, F(ab′), Fv, or scFv), a naturally occurring nucleic acid, an artificial nucleic acid, an aptamer, a peptide aptamer, an oligopeptide, an enzyme, and a coenzyme.

Such a particle for an immunological test that the magnetic particle according to the present invention has a ligand, in which the ligand and the functional group of the polymer layer have a chemical bond therebetween, is a preferred embodiment.

### EXAMPLES

The present invention is described in more detail below by way of Reference Examples, Examples, and Comparative Examples. The present invention is by no means limited to Examples below without departing from the gist of the present invention. “Part(s)” and “%” with regard to the description of the amounts of components are by mass, unless otherwise stated.

### Reference Example 1

(Production of Magnetic Core Particles)

Magnetic core particles in each of which magnetite nanoparticles were associated were produced. First, 1.217 g of iron(III) chloride hexahydrate (FeCl₃·6H₂O; manufactured by Kishida Chemical Co., Ltd.), 2.7 g of sodium acetate (CH₃COONa; manufactured by Kishida Chemical Co., Ltd.), and 0.75 g of polyethylene glycol (average molecular weight: 2,000; PEG2000; manufactured by Kishida Chemical Co., Ltd.) were each dissolved in 10 mL of ethylene glycol (EG; HOCH₂CH₂OH; manufactured by Kishida Chemical Co., Ltd.). CH₃COONa/EG and PEG2000/EG were added in the stated order to FeCl₃·6H₂O/EG to prepare a reaction solution. A container made of glass was used for the preparation of the reaction solution.

Next, the reaction solution was set, together with the container made of glass, in a pressure-resistant container (Taiatsu Techno Corporation) including an internal cylinder made of Teflon (trademark), and was heated in an oven at 180° C. for 24 hours. After the completion of the heating, the resultant was cooled to room temperature, and then the product was washed with each of water and ethanol twice, followed by drying in a dryer set to 60° C. 0.7 g of particles were obtained.

(Analysis of Magnetic Core Particles)

SEM observation of the produced magnetic core particles was performed (S-4800, manufactured by Hitachi High-Technologies Corporation). The long diameters of 100 of the particles were measured at a magnification of 5,000, and as a result, their number average particle diameter was found to be 423 nm. The surfaces of the particles were observed at an increased magnification of 50,000, and as a result, it was able to be recognized that the particles were each an aggregation of nanoparticles each having a particle diameter of 20 nm or less. A typical SEM observation image is shown in FIG. 2.

Next, the crystal structure of the obtained magnetic core particles was analyzed by XRD (manufactured by Panalytical, X′PERT PRO). As a result, it was recognized that the structure was a single layer of magnetite (Fe₃O₄).

### Reference Example 2

Magnetic core particles including a silica layer formed on the surface of each of the magnetic core particles (hereinafter simply referred to as “the silica-coated core particles”) were produced using the magnetic core particles produced in Reference Example 1.

(Formation of Silica Layer)

0.5 g of the magnetic core particles obtained in Reference Example 1 were dispersed in a mixed solution of 75 mL of ethanol (manufactured by Kishida Chemical Co., Ltd.) and 75 mL of pure water. Next, 1.5 mL of TEOS (manufactured by Kishida Chemical Co., Ltd.) was added, 22.5 mL of 28% ammonia water (manufactured by Kishida Chemical Co., Ltd.) was added as a catalyst, and the mixture was subjected to a reaction for 1.5 hours while being stirred. After the reaction, the particles were collected with a neodymium magnet, the solvent was removed, and the residue was washed with pure water 7 times. Part of the particles were dried and subjected to SEM observation, and as a result, the irregularities of the nanoparticles, which had been found in the observation of the surfaces of the magnetic core particles, were found to be absent. Thus, it was recognized that a silica layer had been able to be formed on the surface of each of the magnetic core particles. In addition, the long diameters of 100 of the particles were measured at a magnification of 5,000, and as a result, their number average particle diameter was found to be 479 nm.

### Example 1

(Formation of Polymer Layer)

A polymer layer was formed on each of the silica-coated core particles obtained in Reference Example 2. The particles were dispersed in a mixed solution of 10 mL of ethanol and 10 mL of pure water, and 100 μL of 3-methacryloxypropyltrimethoxysilane (LS-3380, manufactured by Shin-Etsu Chemical Co., Ltd.) was added as a silane coupling agent, followed by thorough mixing. Next, 2 mL of 28% ammonia water was added, and the mixture was stirred for 1.5 hours. Next, the solvent was removed while the particles were collected with a neodymium magnet, followed by thorough washing with pure water. After that, 60 mL of pure water that had been subjected to nitrogen bubbling was added to provide a water dispersion liquid.

Next, the dispersion liquid was placed in a four-necked flask (200 mL), subjected to nitrogen bubbling, and stirred for 15 minutes while being stirred at a stirring speed of 200 rpm. Subsequently, the nitrogen bubbling was switched to a nitrogen flow, and then 50 μL of a styrene monomer (manufactured by Kishida Chemical Co., Ltd.) was added to the dispersion liquid.

Next, 0.02 g of potassium persulfate (manufactured by Sigma-Aldrich) was dissolved in 2 mL of pure water that had been deaerated by nitrogen bubbling in advance, and 1 mL of the solution was added into the flask. Next, heating was performed using an oil bath at 35° C. for 30 minutes, and then the temperature was raised to 60° C. and held for 1 hour. Subsequently, 200 μL of glycidyl methacrylate (manufactured by Kishida Chemical Co., Ltd.) was added, and the whole was further held for 12 hours to complete polymerization. After the completion of the polymerization, the resultant was thoroughly washed with pure water. Thus, particles were obtained.

(Introduction of Functional Group)

Next, treatment for introducing a carboxyl group as a functional group was performed. 10 mg of the obtained particles were dispersed in 5 mL of pure water. Separately from the dispersion liquid, 350 mg of mercaptosuccinic acid (manufactured by Kishida Chemical Co., Ltd.) was dissolved in 5 mL of pure water, and 0.8 mL of triethylamine (manufactured by Tokyo Chemical Industry Co., Ltd.) was added for pH adjustment. 1 mL of the solution was added to the particle dispersion liquid, and the mixture was thoroughly stirred and subjected to heating treatment at 60° C. for 3 hours. After that, the solvent was removed while the particles were collected with a neodymium magnet, followed by thorough washing with pure water. Thus, a magnetic particle dispersion liquid was obtained.

(Number Average Particle Diameter of Magnetic Particles)

SEM observation of the produced magnetic particles was performed. Part of the magnetic particle dispersion solution was dried, and the long diameters of 100 of the particles were measured at a magnification of 5,000, and as a result, their number average particle diameter was found to be 628 nm.

The obtained magnetic particles were analyzed for the constituent components of the polymer layer of the magnetic particles through use of pyrolysis-gas chromatography (PY-3030D manufactured by Frontier Laboratories Ltd.). As a result, signals derived from styrene and glycidyl methacrylate serving as the constituent components of the polymer layer were detected.

### Example 2

(Formation of Polymer Layer)

A polymer layer was formed on each of the silica-coated core particles obtained in Reference Example 2. The particles were dispersed in a mixed solution of 10 mL of ethanol and 10 mL of pure water, and 100 μL of 3-methacryloxypropyltrimethoxysilane (LS-3380, manufactured by Shin-Etsu Chemical Co., Ltd.) was added as a silane coupling agent, followed by thorough mixing. Next, 2 mL of 28% ammonia water was added, and the mixture was stirred for 1.5 hours. Next, the solvent was removed while the particles were collected with a neodymium magnet, followed by thorough washing with pure water. After that, 60 mL of pure water that had been subjected to nitrogen bubbling was added to provide a water dispersion liquid.

Next, the dispersion liquid was placed in a four-necked flask (200 mL), subjected to nitrogen bubbling, and stirred for 15 minutes while being stirred at a stirring speed of 200 rpm. Subsequently, the nitrogen bubbling was switched to a nitrogen flow, and then 50 μL of a styrene monomer (manufactured by Kishida Chemical Co., Ltd.) was added to the dispersion liquid.

Next, 0.02 g of potassium persulfate (manufactured by Sigma-Aldrich) was dissolved in 2 mL of pure water that had been deaerated by nitrogen bubbling in advance, and 1 mL of the solution was added into the flask. Next, heating was performed using an oil bath at 35° C. for 30 minutes, and then the temperature was raised to 60° C. and held for 1 hour. Subsequently, 200 μL of glycidyl methacrylate (manufactured by Kishida Chemical Co., Ltd.) and 200 μL of an acrylic acid monomer (manufactured by Tokyo Chemical Industry Co., Ltd.) were added, and the whole was further held for 12 hours to complete polymerization. After the completion of the polymerization, the resultant was thoroughly washed with pure water. Thus, particles were obtained.

SEM observation of the produced magnetic particles was performed. Part of the magnetic particle dispersion solution was dried, and the long diameters of 100 of the particles were measured at a magnification of 5,000, and as a result, their number average particle diameter was found to be 572 nm.

The obtained magnetic particles were analyzed for the constituent components of the polymer layer of the magnetic particles through use of pyrolysis-gas chromatography as in Example 1. As a result, signals derived from styrene, glycidyl methacrylate, and acrylic acid serving as the constituent components of the polymer layer were detected.

### Example 3

(Formation of Polymer Layer)

A polymer layer was formed on each of the silica-coated core particles obtained in Reference Example 2. The particles were dispersed in a mixed solution of 10 mL of ethanol and 10 mL of pure water, and 100 μL of 3-methacryloxypropyltrimethoxysilane (LS-3380, manufactured by Shin-Etsu Chemical Co., Ltd.) was added as a silane coupling agent, followed by thorough mixing. Next, 2 mL of 28% ammonia water was added, and the mixture was stirred for 1.5 hours. Next, the solvent was removed while the particles were collected with a neodymium magnet, followed by thorough washing with pure water. After that, 60 mL of pure water that had been subjected to nitrogen bubbling was added to provide a water dispersion liquid.

Next, the dispersion liquid was placed in a four-necked flask (200 mL), subjected to nitrogen bubbling, and stirred for 15 minutes while being stirred at a stirring speed of 200 rpm. Subsequently, the nitrogen bubbling was switched to a nitrogen flow, and then 50 μL of a styrene monomer (manufactured by Kishida Chemical Co., Ltd.) and 2.5 μL of a divinylbenzene monomer (manufactured by Kishida Chemical Co., Ltd.) serving as a crosslinking agent were mixed and added to the dispersion liquid.

Next, 0.02 g of potassium persulfate (manufactured by Sigma-Aldrich) was dissolved in 2 mL of pure water that had been deaerated by nitrogen bubbling in advance, and 1 mL of the solution was added into the flask. Next, heating was performed using an oil bath at 35° C. for 30 minutes, and then the temperature was raised to 60° C. and held for 1 hour. Subsequently, 200 μL of glycidyl methacrylate (manufactured by Kishida Chemical Co., Ltd.) and 10 μL of a divinylbenzene monomer (manufactured by Kishida Chemical Co., Ltd.) serving as a crosslinking agent were mixed and added, and the whole was further held for 12 hours to complete polymerization. After the completion of the polymerization, the resultant was thoroughly washed with pure water. Thus, particles were obtained.

(Introduction of Functional Group)

Next, treatment for introducing a carboxyl group as a functional group was performed. 10 mg of the obtained particles were dispersed in 5 mL of pure water. Separately from the dispersion liquid, 350 mg of mercaptosuccinic acid (manufactured by Kishida Chemical Co., Ltd.) was dissolved in 5 mL of pure water, and 0.8 mL of triethylamine (manufactured by Tokyo Chemical Industry Co., Ltd.) was added for pH adjustment. 1 mL of the solution was added to the particle dispersion liquid, and the mixture was thoroughly stirred and subjected to heating treatment at 60° C. for 3 hours. After that, the solvent was removed while the particles were collected with a neodymium magnet, followed by thorough washing with pure water. Thus, a magnetic particle dispersion liquid was obtained.

SEM observation of the produced magnetic particles was performed. Part of the magnetic particle dispersion solution was dried, and the long diameters of 100 of the particles were measured at a magnification of 5,000, and as a result, their number average particle diameter was found to be 619 nm.

The obtained magnetic particles were analyzed for the constituent components of the polymer layer of the magnetic particles through use of pyrolysis-gas chromatography as in Example 1. As a result, signals derived from styrene, glycidyl methacrylate, and divinylbenzene serving as the constituent components of the polymer layer were detected.

### Example 4

(Formation of Polymer Layer)

A polymer layer was formed on each of the silica-coated core particles obtained in Reference Example 2. The particles were dispersed in a mixed solution of 10 mL of ethanol and 10 mL of pure water, and 100 μL of 3-methacryloxypropyltrimethoxysilane (LS-3380, manufactured by Shin-Etsu Chemical Co., Ltd.) was added as a silane coupling agent, followed by thorough mixing. Next, 2 mL of 28% ammonia water was added, and the mixture was stirred for 1.5 hours. Next, the solvent was removed while the particles were collected with a neodymium magnet, followed by thorough washing with pure water. After that, 60 mL of pure water that had been subjected to nitrogen bubbling was added to provide a water dispersion liquid.

Next, the dispersion liquid was placed in a four-necked flask (200 mL), subjected to nitrogen bubbling, and stirred for 15 minutes while being stirred at a stirring speed of 200 rpm. Subsequently, the nitrogen bubbling was switched to a nitrogen flow, and then 50 μL of a styrene monomer (manufactured by Kishida Chemical Co., Ltd.) and 5 μL of a divinylbenzene monomer (manufactured by Kishida Chemical Co., Ltd.) serving as a crosslinking agent were mixed and added to the dispersion liquid.

Next, 0.02 g of potassium persulfate (manufactured by Sigma-Aldrich) was dissolved in 2 mL of pure water that had been deaerated by nitrogen bubbling in advance, and 1 mL of the solution was added into the flask. Next, heating was performed using an oil bath at 35° C. for 30 minutes, and then the temperature was raised to 60° C. and held for 1 hour. Subsequently, 200 μL of glycidyl methacrylate (manufactured by Kishida Chemical Co., Ltd.) and 20 μL of a divinylbenzene monomer (manufactured by Kishida Chemical Co., Ltd.) serving as a crosslinking agent were mixed and added, and the whole was further held for 12 hours to complete polymerization. After the completion of the polymerization, the resultant was thoroughly washed with pure water. Thus, particles were obtained.

(Introduction of Functional Group)

Next, treatment for introducing a carboxyl group as a functional group was performed. 10 mg of the obtained particles were dispersed in 5 mL of pure water. Separately from the dispersion liquid, 350 mg of mercaptosuccinic acid (manufactured by Kishida Chemical Co., Ltd.) was dissolved in 5 mL of pure water, and 0.8 mL of triethylamine (manufactured by Tokyo Chemical Industry Co., Ltd.) was added for pH adjustment. 1 mL of the solution was added to the particle dispersion liquid, and the mixture was thoroughly stirred and subjected to heating treatment at 60° C. for 3 hours. After that, the solvent was removed while the particles were collected with a neodymium magnet, followed by thorough washing with pure water. Thus, a magnetic particle dispersion liquid was obtained.

SEM observation of the produced magnetic particles was performed. Part of the magnetic particle dispersion solution was dried, and the long diameters of 100 of the particles were measured at a magnification of 5,000, and as a result, their number average particle diameter was found to be 604 nm.

The obtained magnetic particles were analyzed for the constituent components of the polymer layer of the magnetic particles through use of pyrolysis-gas chromatography as in Example 1. As a result, signals derived from styrene, glycidyl methacrylate, and divinylbenzene serving as the constituent components of the polymer layer were detected. The signal derived from divinylbenzene was increased as compared to the particles of Example 3.

### Example 5

(Formation of Polymer Layer)

A polymer layer was formed on each of the silica-coated core particles obtained in Reference Example 2. The particles were dispersed in a mixed solution of 10 mL of ethanol and 10 mL of pure water, and 100 μL of 3-methacryloxypropyltrimethoxysilane (LS-3380, manufactured by Shin-Etsu Chemical Co., Ltd.) was added as a silane coupling agent, followed by thorough mixing. Next, 2 mL of 28% ammonia water was added, and the mixture was stirred for 1.5 hours. Next, the solvent was removed while the particles were collected with a neodymium magnet, followed by thorough washing with pure water. After that, 60 mL of pure water that had been subjected to nitrogen bubbling was added to provide a water dispersion liquid.

Next, the dispersion liquid was placed in a four-necked flask (200 mL), subjected to nitrogen bubbling, and stirred for 15 minutes while being stirred at a stirring speed of 200 rpm. Subsequently, the nitrogen bubbling was switched to a nitrogen flow, and then 50 μL of a styrene monomer (manufactured by Kishida Chemical Co., Ltd.) and 5 μL of trimethylolpropane trimethacrylate (manufactured by Tokyo Chemical Industry Co., Ltd.) serving as a crosslinking agent were mixed and added to the dispersion liquid.

Next, 0.02 g of potassium persulfate (manufactured by Sigma-Aldrich) was dissolved in 2 mL of pure water that had been deaerated by nitrogen bubbling in advance, and 1 mL of the solution was added into the flask. Next, heating was performed using an oil bath at 35° C. for 30 minutes, and then the temperature was raised to 60° C. and held for 1 hour. Subsequently, 200 μL of glycidyl methacrylate (manufactured by Kishida Chemical Co., Ltd.) and 20 μL of trimethylolpropane trimethacrylate (manufactured by Tokyo Chemical Industry Co., Ltd.) serving as a crosslinking agent were mixed and added, and the whole was further held for 12 hours to complete polymerization. After the completion of the polymerization, the resultant was thoroughly washed with pure water. Thus, particles were obtained.

(Introduction of Functional Group)

Next, treatment for introducing a carboxyl group as a functional group was performed. 10 mg of the obtained particles were dispersed in 5 mL of pure water. Separately from the dispersion liquid, 350 mg of mercaptosuccinic acid (manufactured by Kishida Chemical Co., Ltd.) was dissolved in 5 mL of pure water, and 0.8 mL of triethylamine (manufactured by Tokyo Chemical Industry Co., Ltd.) was added for pH adjustment. 1 mL of the solution was added to the particle dispersion liquid, and the mixture was thoroughly stirred and subjected to heating treatment at 60° C. for 3 hours. After that, the solvent was removed while the particles were collected with a neodymium magnet, followed by thorough washing with pure water. Thus, a magnetic particle dispersion liquid was obtained.

SEM observation of the produced magnetic particles was performed. Part of the magnetic particle dispersion solution was dried, and the long diameters of 100 of the particles were measured at a magnification of 5,000, and as a result, their number average particle diameter was found to be 598 nm.

The obtained magnetic particles were analyzed for the constituent components of the magnetic particles through use of pyrolysis-gas chromatography as in Example 3. As a result, signals derived from styrene, glycidyl methacrylate, and trimethylolpropane trimethacrylate serving as the constituent components of the polymer layer were detected.

### Example 6

(Formation of Polymer Layer)

A polymer layer was formed on each of the silica-coated core particles obtained in Reference Example 2. The particles were dispersed in a mixed solution of 10 mL of ethanol and 10 mL of pure water, and 100 μL of 3-methacryloxypropyltrimethoxysilane (LS-3380, manufactured by Shin-Etsu Chemical Co., Ltd.) was added as a silane coupling agent, followed by thorough mixing. Next, 2 mL of 28% ammonia water was added, and the mixture was stirred for 1.5 hours. Next, the solvent was removed while the particles were collected with a neodymium magnet, followed by thorough washing with pure water. After that, 60 mL of pure water that had been subjected to nitrogen bubbling was added to provide a water dispersion liquid.

Next, the dispersion liquid was placed in a four-necked flask (200 mL), subjected to nitrogen bubbling, and stirred for 15 minutes while being stirred at a stirring speed of 200 rpm. Subsequently, the nitrogen bubbling was switched to a nitrogen flow, and then 50 μL of a styrene monomer (manufactured by Kishida Chemical Co., Ltd.) and 5 μL of polyethylene glycol diacrylate (manufactured by Tokyo Chemical Industry Co., Ltd.) serving as a crosslinking agent were mixed and added to the dispersion liquid.

Next, 0.02 g of potassium persulfate (manufactured by Sigma-Aldrich) was dissolved in 2 mL of pure water that had been deaerated by nitrogen bubbling in advance, and 1 mL of the solution was added into the flask. Next, heating was performed using an oil bath at 35° C. for 30 minutes, and then the temperature was raised to 60° C. and held for 1 hour. Subsequently, 200 μL of glycidyl methacrylate (manufactured by Kishida Chemical Co., Ltd.) and 20 μL of polyethylene glycol diacrylate (manufactured by Tokyo Chemical Industry Co., Ltd.) serving as a crosslinking agent were mixed and added, and the whole was further held for 12 hours to complete polymerization. After the completion of the polymerization, the resultant was thoroughly washed with pure water. Thus, particles were obtained.

(Introduction of Functional Group)

Next, treatment for introducing a carboxyl group as a functional group was performed. 10 mg of the obtained particles were dispersed in 5 mL of pure water. Separately from the dispersion liquid, 350 mg of mercaptosuccinic acid (manufactured by Kishida Chemical Co., Ltd.) was dissolved in 5 mL of pure water, and 0.8 mL of triethylamine (manufactured by Tokyo Chemical Industry Co., Ltd.) was added for pH adjustment. 1 mL of the solution was added to the particle dispersion liquid, and the mixture was thoroughly stirred and subjected to heating treatment at 60° C. for 3 hours. After that, the solvent was removed while the particles were collected with a neodymium magnet, followed by thorough washing with pure water. Thus, a magnetic particle dispersion liquid was obtained.

SEM observation of the produced magnetic particles was performed. Part of the magnetic particle dispersion solution was dried, and the long diameters of 100 of the particles were measured at a magnification of 5,000, and as a result, their number average particle diameter was found to be 622 nm.

The obtained magnetic particles were analyzed for the constituent components of the polymer layer of the magnetic particles through use of pyrolysis-gas chromatography as in Example 3. As a result, signals derived from styrene, glycidyl methacrylate, and polyethylene glycol diacrylate serving as the constituent components of the polymer layer were detected.

### Reference Example 3

Magnetic core particles in each of which magnetite nanoparticles were associated were produced. The magnetic core particles were produced by the same procedure as in Reference Example 1 except that 0.30 g of iron(II) chloride tetrahydrate (FeCl₂·4H₂O, manufactured by Kishida Chemical Co., Ltd.) and 0.81 g of iron(III) chloride hexahydrate (FeCl₃·6H₂O, manufactured by Kishida Chemical Co., Ltd.) were used as raw materials for magnetite.

SEM observation of the produced magnetic core particles was performed. The long diameters of 100 of the particles were measured at a magnification of 5,000, and as a result, their number average particle diameter was found to be 519 nm.

### Comparative Example 1

1.35 g of styrene and 0.15 g of divinylbenzene serving as a monomer and crosslinking agent for forming particles, and 0.06 g of 2,2′-azobisisobutyronitrile were added to and mixed with 14 g of EMG1400 (manufactured by Ferrotec) serving as magnetite nanoparticles to produce a monomer mixed liquid. Next, 75 mL of an aqueous solution having dissolved therein 0.75 g of sodium dodecyl sulfate was added to the resultant monomer mixed liquid, and the mixture was subjected to ultrasonic dispersion treatment (10 repetitions of 2 minutes of ultrasonic irradiation (ultrasonic power: 150 W) and 2 minutes of subsequent suspension of ultrasonic irradiation) under ice cooling through use of an ultrasonic homogenizer (UD-200, manufactured by Tomy Seiko Co., Ltd.).

Next, the resultant emulsion was subjected to a polymerization reaction at 70° C. for 7 hours, followed by collection with a neodymium magnet and thorough washing with pure water. Thus, magnetic particles each having magnetite dispersed in a polymer were obtained.

(Analysis of Magnetic Particles)

The particle diameter size of 100 of the obtained magnetic particles according to SEM observation was about 100 nm. In addition, the weight loss of the magnetic particles by heating at 500° C. was analyzed with a thermal analyzer (TGA manufactured by Hitachi High-Technologies Corporation), and as a result, it was found that the magnetic particles contained 13 mass % of a polymer component.

SEM observation of the produced magnetic particles was performed. The long diameters of 100 of the particles were measured at a magnification of 5,000, and as a result, their number average particle diameter was found to be 687 nm.

### Comparative Example 2

2.7 g of iron(III) chloride hexahydrate and 1.0 g of iron(II) chloride tetrahydrate were dissolved in 375 mL of water in a reaction vessel, and while the whole was stirred, a solution obtained by mixing 4 mL of 28% ammonia water and 100 mL of water was added dropwise over 1 hour. After the dropwise addition, the mixture was stirred for 1 hour, and the temperature was raised to 80° C. After that, 10.5 g of oleic acid was added, and stirring was continued for 2 hours. After cooling to room temperature, magnetite particles having oleic acid adsorbed thereon, which had been obtained by being collected with a neodymium magnet, were thoroughly washed with pure water. 5.7 mL of decane and 2.2 mL of TEOS were added to and mixed with the obtained magnetite particles having oleic acid adsorbed thereon to prepare a dispersion liquid (A).

39.0 mL of a 28% aqueous ammonia solution, 55.4 mL of isopropanol, 2.9 mL of sorbitan monooleate (manufactured by FUJIFILM Wako Pure Chemical Corporation), and 2.0 mL of a polyoxyethylene alkyl ether (manufactured by Sanyo Chemical Industries, Ltd.) were loaded into a reaction vessel and mixed using a homogenizer (UD-200, manufactured by Tomy Seiko Co., Ltd.). After the temperature had been raised to 50° C., the above-mentioned dispersion liquid (A) was added dropwise over 1 hour, and then the mixture was subjected to a reaction at 50° C. for 1 hour. The reaction was followed by collection with a neodymium magnet and removal of a supernatant in which uncollectible fine particles remained. The resultant particles were thoroughly washed with pure water. Thus, magnetic silica particles were obtained.

(Analysis of Magnetic Particles)

The produced magnetic particles were analyzed for their crystal structure by XRD. As a result, a diffraction image in which a peak of magnetite (Fe₃O₄) and a broad peak of amorphous silica coexisted was obtained. Thus, it was recognized that the structure was a composite layer of magnetite and silica.

In addition, the particle diameter size of 100 of the magnetic particles according to SEM observation was about 488 nm.

(Comparison in Magnetic Field Responsiveness)

The particles of Reference Example 2, Examples 1 and 2, and Comparative Examples 1 and 2 were evaluated for their differences in magnetic field responsiveness. 50 mg of the particles of each kind were mixed into 40 mL of pure water and dispersed in a screw-capped tube made of glass having a volume of 50 mL. Next, a columnar neodymium magnet (φ30 mm×t18 mm, manufactured by NeoMag Co., Ltd.) was placed at the bottom of the screw-capped tube, and periods of time taken for the completion of the capture of the magnetic particles were compared (Table 1). It was recognized that the magnetic particles of the present invention had high magnetic field responsiveness as compared to the particles of the related-art structures.

TABLE 1

| | Amount of | Amount of | Particle | Capture |
|---|---|---|---|---|
| | particles | dispersion | size | time |
| | (mg) | solution (mL) | (nm) | (seconds) |
| Reference | 50 | 40 | 479 | 57 |
| Example 2 | | | | |
| Example 1 | | | 628 | 89 |
| Example 2 | | | 572 | 76 |
| Example 3 | | | 619 | 86 |
| Example 4 | | | 604 | 84 |
| Example 5 | | | 598 | 80 |
| Example 6 | | | 622 | 88 |
| Comparative | | | 687 | 118 |
| Example 1 | | | | |
| Comparative | | | 488 | 103 |
| Example 2 | | | | |

According to the present invention, the magnetic particle that enables the magnetic particle that has captured a substance of interest, such as an antigen or an antibody, in a specimen solution, to be quickly collected with a magnetic field, and that can be quickly redispersed after the magnetic field is stopped can be provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-098318, filed Jun. 17, 2022, and Japanese Patent Application No. 2023-086755, filed May 26, 2023, which are hereby incorporated by reference herein in their entirety.

What is claimed is:

1. A magnetic particle comprising:
a magnetic core particle; and
a polymer layer arranged on a surface of the magnetic core particle,
wherein the magnetic core particle contains an aggregation of a plurality of magnetic nanoparticles, and
wherein the polymer layer contains a polymer having at least one kind of functional group selected from the group consisting of: a carboxyl group; an amino group; a thiol group; an epoxy group; a maleimide group; and a succinimidyl group.

2. The magnetic particle according to claim 1, wherein the magnetic nanoparticles have a number average particle diameter of 20 nm or less, and each contain a magnetic iron oxide.

3. The magnetic particle according to claim 2, wherein the magnetic iron oxide is formed of at least any one of magnetite (Fe₃O₄) or maghemite (γ-Fe₂O₃).

4. The magnetic particle according to claim 1, wherein the polymer layer is formed on the surface of the magnetic core particle via a silica layer.

5. The magnetic particle according to claim 1, wherein the polymer layer includes at least a first polymer layer and a second polymer layer.

6. The magnetic particle according to claim 5, wherein the first polymer layer contains a hydrophobic polymer.

7. The magnetic particle according to claim 5, wherein the second polymer layer formed on the first polymer layer and serving as an outermost surface layer of the magnetic particle contains a polymer having a unit derived from glycidyl methacrylate.

8. The magnetic particle according to claim 1, wherein the magnetic particle has a number average particle diameter of 0.1 μm or more and 2.0 μm or less.

9. The magnetic particle according to claim 1, wherein the functional group is capable of being bonded to a ligand.

10. A particle for an immunological test comprising:
the magnetic particle according to claim 1; and
a ligand,
wherein the ligand and the functional group have a chemical bond therebetween.

11. The particle for an immunological test according to claim 10, wherein the ligand is one of an antibody or an antigen.
